(19) United States
(12) Patent Application Publication
(10) Pub. No.: US 2007/0129791 A1
(43) Pub. Date: Jun. 7, 2007

(54) Stent with integral filter

(76) Inventor: Malur R. Balaji, Pittsford, NY (US)

Correspondence Address: BURNS & LEVINSON, LLP, 125 SUMMER STREET, BOSTON, MA, 02110, US

(21) Appl. No.: 11/486,168

(22) Filed: Jul. 13, 2006

Related U.S. Application Data
(60) Provisional application No. 60/742,917, filed on Dec. 5, 2005.
(60) Provisional application No. 60/742,315, filed on Dec. 5, 2005.

(51) Int. Cl. A61F 2/06

(52) U.S. Cl. 623/1.44

(57) ABSTRACT

Apparatus and methods for protecting against embolization.

### CROSS REFERENCE TO RELATED APPLICATIONS

This application claims priority of U.S. Provisional Application 60/742,917 entitled STENT WITH INTEGRAL FILTER”, filed on Dec. 5, 2005, and of U.S. Provisional Application 60/742,315, entitled STENTS WITH BEVELED ENDS AND METHODS OF THE USE THEREOF, also filed on Dec. 5, 2005, both of which are hereby incorporated by reference herein.

### BACKGROUND

The teachings provided herein relate to the apparatus and methods for protecting against embolization.

Narrowing of an artery may be caused by the accumulation of plaque (e.g., fatty deposits, calcium, cell debris) deposits on the intima (inner lining) of the artery (as shown in FIG. 1). Stenting is the permanent placement of a small, latticed tube inside the artery to provide structural support and to keep the lumen (hollow channel) open to maintain blood flow.

The stenting procedure involves passing a collapsed stent into the artery to the site that requires support. The lattices of the stent are then allowed to expand, increasing the diameter of the stent. The expanded stent is then left permanently in place in the vessel.

Various means have been described to deliver and implant stents. One method frequently described for delivering a stent to a desired intraluminal location includes mounting the expandable stent on an expandable member, such as a balloon, provided on the distal end of an intravascular catheter, advancing the catheter to the desired location within the patient's body lumen, inflating the balloon on the catheter to expand the stent into a permanent expanded condition and then deflating the balloon and removing the catheter.

The current design of vascular stents is a tube whose wall is constructed of an expandable, structural, open-lattice made of a material such as nickel titanium (NiTinol), stainless steel, or other materials. Due to the open-lattice design, plaque from the wall of the artery may squeeze through the interstices (openings) of the lattice and protrude the lumen (hollow channel) of the artery (as shown in FIG. 2). The protruding atheroma (plaque material) may obstruct blood flow. Particles of the protruding atheroma may break loose and travel freely in the bloodstream posing a risk of embolization.

The current method for protecting against embolization caused by the release of plaque particles during the deployment of a stent is the use of a separate embolic protection device. The separate embolic protection filter is passed via catheter through the narrowed site of the artery and opened downstream of the site. The filter is intended to collect any particles that may be released when the stent is opened at the narrowed site. After the stent is in place, the filter and its contents are removed. This is typically accomplished by retracting the catheter and embolic protection filter through the stent and the artery. Once the filter is removed, it provides no further protection for entrapping emboli that originate from the site of the stent. That is, because the filter is no longer present, it cannot entrap emboli that may be created by portions of the atheroma that squeeze through the interstices of the stent, protrude into the lumen of the stent, and then break free from the stent and flow in the bloodstream.

Deploying and retrieving the separate embolic protection filter pose known risks. Accidental separation of the filter basket from the guide catheter has contributed to adverse events including unplanned surgery, TIA, occultation, and death. The following factors have been identified as potential contributors to these adverse events:
Loss of access to the guiding catheter from the common carotid artery (CCA) after the filter has been deployed
Pulling an open filter into the stent resulting in entanglement of the two devices
Difficulty navigating the recovery catheter through the deployed stent, leading to either of the two prior problems
Fracture of the guide wire of the separate embolic protection filter.

In addition, the initial placement of the separate embolic protection filter requires that the catheter and filter be passed through the narrowed site of the artery before the filter basket can be opened. The movement of the catheter and filter through the narrowed site can disrupt plaque or other debris along the intima of the artery releasing them as emboli. Furthermore, retrieving the embolic protection filter through the stent may dislodge portions or the atheroma that protrude through the stent interstices and into the lumen of the stent. The dislodged portions or the atheroma may be released in to the bloodstream as emboli (see FIGS. 1a, 1b, 2a, 2b).

There is therefore a need to provide methods and apparatus for protecting against embolization during endovascular procedures that do not include the risk of separation of the filter.

There is a further need to provide methods and apparatus for protecting against embolization during endovascular procedures that minimize the release of emboli.

### BRIEF SUMMARY

In one embodiment, the stent of these teachings includes a substantially cylindrical expandable structure comprising a plurality of interconnected elements and a filtering mesh attached to at least some of the interconnected elements. The filter mesh may be attached to the outside surface of the stent, attached to the inside surface of the stent, woven among and within the lattices of the stent, or any combination of inside, outside or among the lattices of the stent.

Methods for using the stent of these teachings in order to protect from embolization are also disclosed.

For a better understanding of the present teachings, together with other and further needs thereof, reference is made to the accompanying drawings and detailed description and its scope will be pointed out in the appended claims.

### BRIEF DESCRIPTION OF THE SEVERAL VIEWS OF THE DRAWING

FIGS. 1a, 1b show a vessel with plaque deposits;

FIGS. 2a, 2b show a vessel with a conventional stent;

FIGS. 3a, 3b show a vessel with the stent of these teachings; FIG. 3a presents the cross sectional view and FIG. 3b presents the side view;

FIGS. 4a, 4b show the structural lattice included in the stent of these teachings; FIG. 4a presents the cross sectional view and FIG. 4b presents the side view;

FIGS. 5a, 5b, 5c, 5d represent a cross section and lateral view of embodiments of the stent of these teachings;

FIG. 6 illustrates a stent incorporating features of these teachings which is mounted onto a delivery catheter;

FIG. 7 shows an embodiment of the stent of these teachings holding open an artery;

FIG. 8 shows another embodiment of the stent of these teachings holding open an artery; and

FIG. 9 depicts an application of an embodiment of the stent of these teachings.

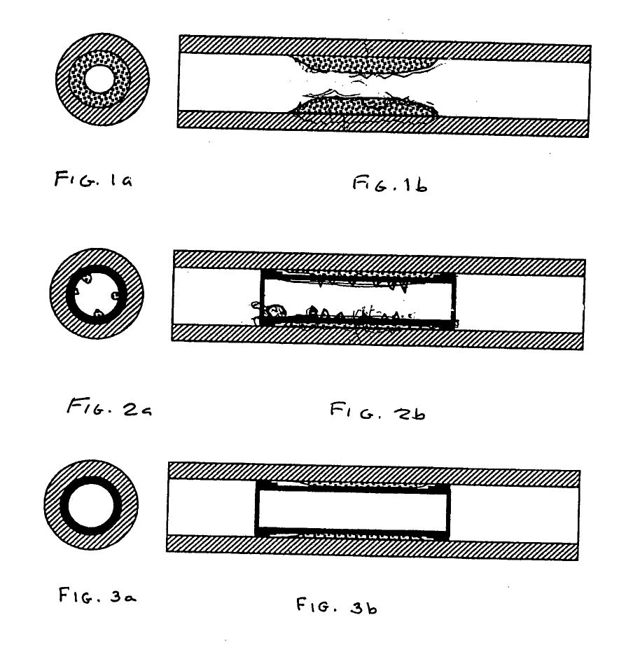

### DETAILED DESCRIPTION

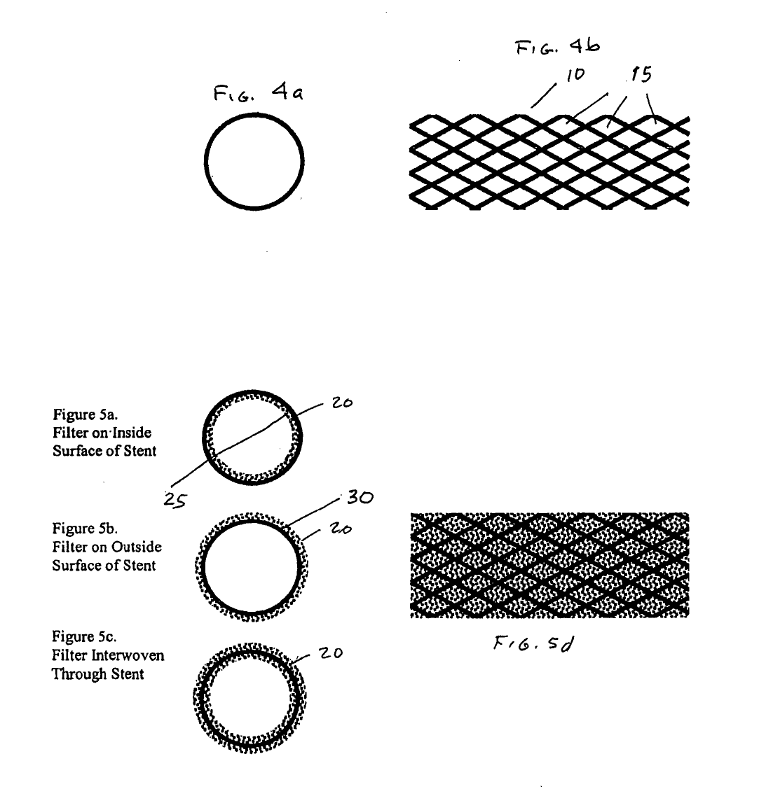

In one embodiment, the stent of these teachings includes a substantially cylindrical expandable structure (10, FIG. 4b) comprising a plurality of interconnected elements (lattice) (15, FIG. 4b) and a filter mesh (20, FIGS. 5a, 5b, 5c) attached to at least some of the interconnected elements. The filter mesh 20 is a fine mesh with the ability to filter particles that are of clinically significant size. (The filter mesh 20 of these teachings should be distinguished from a generally tubular body having a number of openings in relatively thin-walled regions of the tubular body.) The filter mesh 20 may be attached to the outside surface of the substantially cylindrical expandable structure, attached to the inside surface of the substantially cylindrical expandable structure, woven among and within the lattices of the substantially cylindrical expandable structure, or any combination of inside, outside or among the lattices of the substantially cylindrical expandable structure. The structural lattice included in the stent of these teachings (such as that shown in FIGS. 4a, 4b) can be a lattice such as, but not limited to, the lattice disclosed in U.S. Pat. No. 6,432,133 and in U.S. Pat. No. 5,354,308, both of which are incorporated by reference herein.

The filter mesh 20 of these teachings can be fabricated from a variety of different materials, such as but not limited to, a woven or braided plastic or metallic mesh, a Nitinol mesh, combinations thereof, or other material that is capable of providing a mesh to capture material within flowing blood, while allowing the blood to flow through the pores or apertures thereof. Generally, the filter mesh of these teachings can be fabricated from a variety of materials so long as the filter mesh is capable of being operatively connected to at least some of the interconnected elements and is bio-compatible.

The filter mesh of these teachings is capable of receiving a variety of different coatings, applied to the elements constituting the mesh. The different coatings can be used, for example, but are not limited to, for improving lubricity, for providing anti-thrombogenic properties, and/or for reducing platelet aggregation. These coatings can include, but are not limited to, a hydrophilic coating, a heparinized coating, Teflon, silicone, or other coating known to those skilled in the art in light of the teaching contained herein. The substantially cylindrical expandable structure is also capable of receiving a variety of different coatings (including synthetic coatings), applied to the lattice.

In one embodiment, the filter mesh 20 of these teachings is a wire mesh. (In the embodiment in which the filter mesh of these teachings is a wire mesh, any coating would be applied to individual wires.) The wire mesh should be formed of a material which is both resilient and can be treated to substantially set a desired shape.

The wire mesh of these teachings can be formed of a material which is both resilient and can be heat treated to substantially set a desired shape. Materials which are believed to be suitable for this purpose include a cobalt-based low thermal expansion alloy referred to in the field as Elgiloy, nickel based high-temperature high-strength “superalloys” commercially available from Haynes International under the trade name Hastelloy, nickel-based heat treatable alloys sold under the name Incoloy by International Nickel, and a number of different grades of stainless steel. The important factor in choosing a suitable material for the wires is that the wires retain a suitable amount of the deformation induced by the molding surface (as described below) when subjected to a predetermined heat treatment.

One class of materials which meet these qualifications is the class of so-called shape memory alloys. Shape memory alloys are a group of metallic materials that demonstrate the ability to return to a defined shape or size when subjected to certain thermal or stress conditions. Shape memory alloys are generally capable of being plastically deformed at a relatively low temperature and, upon exposure to a relatively higher temperature, return to the defined shape or size prior to the deformation. Shape memory alloys may be further defined as one that yields a thermoplastic martensite. A shape memory alloy which yields a thermoplastic martensite undergoes a martensitic transformation of a type that permits the alloy to be deformed by a twinning mechanism below the martensitic transformation temperature. The deformation is then reversed when the twinned structure reverts upon heating to the parent austenite phase. The austenite phase occurs when the material is at a low strain state and occurs at a given temperature. The martensite phase may be either temperature-induced martensite (TIM) or stress-induced martensite (SIM).

One particularly preferred shape memory alloy for use in the present method is NiTinol, an approximately stoichiometric alloy of nickel and titanium, which may also include other minor amounts of other metals to achieve desired properties. NiTi alloys such as NiTinol, including appropriate compositions and handling requirements, are well known in the art and such alloys need not be discussed in detail here. For example, U.S. Pat. Nos. 5,067,489 (Lind) and 4,991,602 (Amplatz et al.), the teachings of which are incorporated herein by reference, discuss the use of shape memory NiTi alloys in guidewires. Such NiTi alloys are preferred, at least in part, because they are commercially available and more is known about handling such alloys than other known shape memory alloys. The shape memory alloy can return to a preset expanded configuration for deployment.

In one embodiment, shown in FIG. 5c, the filter mesh is woven, such that a material is integrally woven with a stent material, so that a resulting stent is formed having the filter integrally provided with the stent.

In another embodiment, shown in FIGS. 5a and 5b, the filter mesh 20 is attached to the inside (25, FIG. 5a) or outside surface (30, FIG. 5b) of the substantially cylindrical expandable structure 10. The filter mesh 20 can be attached by means of a variety of attachment means such as, but not limited to, wire clamp structures, or by soldering, brazing, or welding, or otherwise affix the filter mesh to interconnected elements (e.g. with a biocompatible cementitious organic material) to at least some elements of the lattice of the substantially cylindrical expandable structure.

In yet another embodiment, the embodiment shown in FIGS. 5a and 5b is obtained by a three dimensional extension of a filter mesh lattice such as, but not limited to, the lattice disclosed in U.S. Pat. No. 6,432,133 and in U.S. Pat. No. 5,354,308, in which a finer filter mesh lattice is attached to the structural lattice. (A side view of the stent of these teachings is shown in FIG. 5d.)

The substantially cylindrical expandable structure of the stent of these teachings may be constructed of any material that permits the structure to be expandable, rigid upon expansion, and that is compatible with the use as a stent in a vessel of the human body.

In one embodiment, the substantially cylindrical expandable structure is fabricated of a shape memory alloy, which is encapsulated in its final diametric dimension, and the encapsulated substantially cylindrical expandable structure is manipulated into its reduced diametric dimension and radially expanded in vivo under the influence of a transformation.

In the embodiment in which the filter mesh is fabricated of a shape memory alloy, the filter mesh can be encapsulated in its final diametric dimension, and the encapsulated filter mesh is manipulated into its reduced diametric dimension and radially expanded in vivo under the influence of a transformation.

In one embodiment of the method of these teachings, providing a stent comprising a substantially cylindrical expandable structure having a plurality of interconnected elements and a filter mesh operatively connected to at least some of elements from the plurality of interconnected elements is provided and, subsequently, delivered to a predetermined intraluminal location.

In one embodiment of the method of these teachings, the stent of these teachings is passed via catheter into the narrowed site of the artery using a method and devices such as, but not limited to, that described in U.S. Pat. No. 6,432,133, which is incorporated by reference herein. There it is expanded and left permanently in place. Since the filter is integral to the stent, the filter is provided by the insertion of the stent of these teachings (as shown in FIG. 3).

In one instance, the method for delivering a stent of these teachings to a desired intraluminal location includes mounting the expandable stent on an expandable member, such as a balloon, provided on the distal end of an intravascular catheter, introducing the delivery catheter with the stent mounted on the inflatable balloon within a patience vasculature, advancing the catheter over a guide wire to the desired (predetermined) location within the patient's body lumen, inflating the balloon on the catheter to expand the stent into a permanent expanded condition and then deflating the balloon and removing the catheter.

FIG. 6 illustrates a stent 40 incorporating features of the teachings which is mounted onto a delivery catheter 41. In one instance, such as that described in U.S. Pat. No. 6,432,133, which is incorporated by reference herein, the stent of these teachings not being limited to this instance, the stent includes a plurality of radially expandable cylindrical interconnected elements. The delivery catheter 41 has an expandable portion or balloon 44 for expanding of the stent 40 within an artery 55. The artery 55, as shown in FIG. 6 has a dissected lining 56 which has occluded a portion of the arterial passageway.

The delivery catheter 41 onto which the stent 40 is mounted, is essentially the same as a conventional balloon dilatation catheter for angioplasty procedures. The balloon 44 may be formed of suitable materials such as, but not limited to, polyethylene, polyethylene terephthalate, polyvinyl chloride, nylon and ionomers such as Surlyn™ manufactured by the Polymer Products Division of the Du Pont Company. Other polymers may also be used. In order for the stent 40 to remain in place on the balloon 44 during delivery to the site of the damage within the artery 55, the stent 40 is compressed onto the balloon. One embodiment of a retractable protective delivery sleeve 50, such as, but not limited to, that described in U.S. Pat. No. 5,507,768, entitled STENT DELIVERY SYSTEM, which is incorporated by reference herein, may be provided to further ensure that the stent stays in place on the expandable portion of the delivery catheter 41 and prevent abrasion of the body lumen by the open surface of the stent 40 during delivery to the desired arterial location. Other means for securing the stent 40 onto the balloon 44 may also be used, such as providing collars or ridges on the ends of the working portion, i.e. the cylindrical portion, of the balloon.

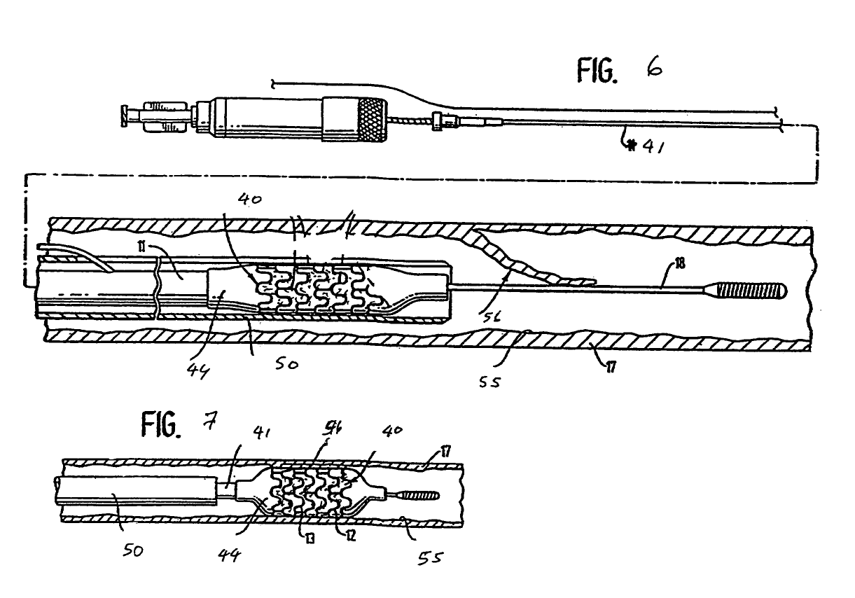

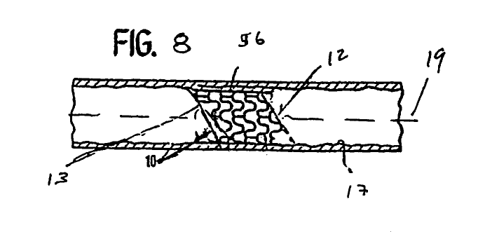

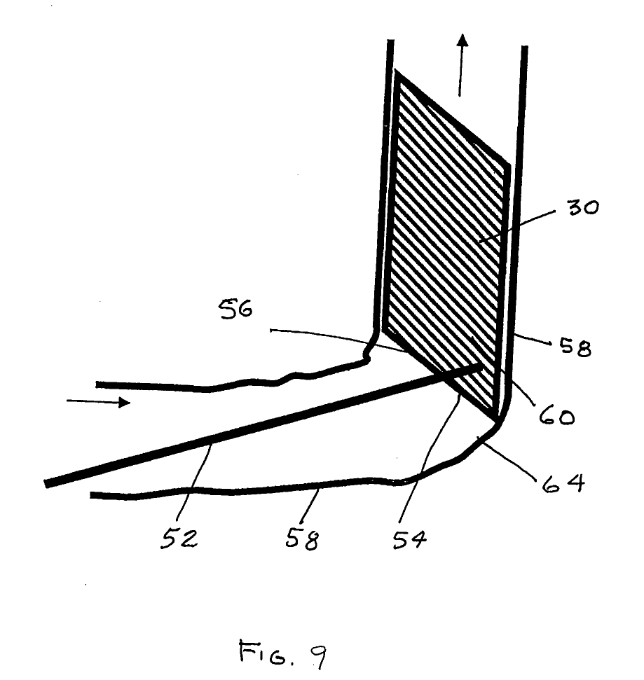

FIG. 7 shows an embodiment of the stent 40 of these teachings holding open the artery 17 after the catheter 41 is withdrawn. Referring to FIG. 7, the stent 40 includes a substantially cylindrical expandable structure.

The filter mesh of these teachings, which is integrated with the structural lattices of the stent, confines the plaque between the wall of the vessel and the wall of the stent when the stent expands. The integral filter of these teachings prevents plaque from protruding through the interstices of the lattice of the stent and from becoming emboli.

In the embodiment in which the integral filter of these teachings is constructed from a nickel titanium mesh, the integral filter more biologically compatible and less thrombogenic (less prone to the formation of clots) than other embodiments constructed of dacron or PTFE (PolyTetraFluoroEthylene) fibers. The improved biologically compatibility is due to the superior porosity of the nickel titanium mesh compared to meshes constructed from dacron or PTFE fibers.

In one embodiment, the filter mesh of these teachings can have a variety of differently sized mesh openings 51 ranging typically, but not limited to, less than about 400 microns (in one instance, less than about 200 microns).

Furthermore, the integral filter of these teachings does not prevent or preclude the use of a separate embolic protection filter or balloon angioplasty when the stent is installed. For example in the case of a tight stenosis, an embolic protection filter and a balloon angioplasty may be deployed before the stent is deployed. Once the stent is in place, the embolic protection filter and a balloon angioplasty may be retracted and retrieved through the lumen of the stent.

The method of these teachings for protecting against embolization includes the deployment the stent of these teachings in a manner that may reduce or eliminate the need for a separate filter for embolic protection because the plaque debris is entrapped at the stent before it can become emboli. Elimination of a separate filter for embolic protection is desirable because deployment and retrieval of the separate filter poses its own risks, including the disruption of plaque debris that may form emboli. The deployment of the stent of these teachings also provides permanent protection against the protrusion of atheroma through the interstices of the stent into the lumen.

The filter of these teachings entraps plaques debris between the intima (inner lining) of the artery and the wall of the stent when the stent is deployed. Because the filter remains permanently in place, it also provides permanent protection for preventing the atheroma (plaque debris and deposits) from growing through the interstices of the stent into the lumen (hollow channel) of the stent where the protruding atheroma may (a) partially obstruct the blood flow, (b) provide surfaces that collect additional plaque debris that contribute to thrombosis, and (c) break-off into particles that flow freely in the blood stream as emboli.

The integral filter of these teachings:
1.) Entraps the atheromatous plaque and debris at the site of the stent, thereby preventing emboli from flowing freely into the bloodstream
2.) Prevents protrusion of atheroma from the intima (inner lining) of an artery through the interstices (spaces) of the stent into the lumen (hollow channel) of the artery where it might impede blood flow or break free as emboli;
3.) Eliminates the need for—but does not preclude the use of—a separate embolic protection filter during stenting;
4.) When constructed from nickel titanium mesh, the integral filter is more biologically compatible and less thrombogenic than filters constructed of dacron or PTFE fibers 5.) An embodiment of the stent of these teachings may reduce the incidence of recurrent stenosis by isolating the atheroma from the main blood stream, as exposure of the atheroma to the blood stream may promulgate further growth of the atheromatous bulge into the lumen of the stented area of the artery.

In one embodiment, the stent of these teachings includes a substantially cylindrical expandable structure having two ends, a locus of points at one of the two ends defining a surface, the surface being beveled with respect to a central axis of the substantially cylindrical expandable structure.

Referring again to FIG. 6, an embodiment 40 of the stent of these teachings, which incorporates at least one beveled end, is mounted onto the delivery catheter 41.

FIG. 8 shows an embodiment of the stent 40 of these teachings holding open the artery 17 after the catheter 411 is withdrawn. Referring to FIG. 8, the stent 40 is a substantially cylindrical expandable structure having two ends 12, 13. A locus of points at one of the two ends 12, 13 defines a surface, which is beveled with respect to a central axis 19 of the stent 40. In the embodiment shown in FIG. 8, both ends 12, 13 define surfaces that are beveled. In the instance in which both surfaces are beveled, embodiments in which an angle between a normal to the first beveled surface and the central axis is different from an angle between a normal to the other beveled surface and the central axis are within the scope of these teachings.

In some instances, in conventional stent designs, the insertion of a catheter into the lumen of a stented vessel is difficult. The perpendicular end of a stent creates an abrupt transition between the vessel and the stent. A catheter inserted into the artery will encounter the pointed tips of the conventional stent lattices at the same position along the length of the artery. A common practice for cannulation is to use an angled catheter that can be rotated to move the distal end of the catheter away from obstructions, including the tips of the lattices located at the proximal end of the stent. However, the perpendicular cut of conventional stent increases the likelihood that the distal end of the catheter will collide with the proximal end of one or more lattices—even when the catheter is rotated. That is, the perpendicular cut of conventional stents creates an obstacle that challenges the current practices of cannulation. Great difficulty may be encountered when attempting to pass a catheter through the site because the catheter may be unable to turn within the tight radius, or the end of the catheter may become caught on the proximal edge of the conventional stent, or the end of the catheter may become caught between the outer surface of the conventional stent and the inner surface of the artery.

As shown in FIG. 9, an embodiment of the stent 40 of these teachings allows an angled catheter 52 to be placed into the proximal end 54 of the stent 40 with less difficulty than stents that feature perpendicular ends. The diagonal cut of the beveled end 56 of stent of this invention 40 provides a more gradual transition between the vessel 58 and the stent 40. This beveled end 56 allows a catheter 52 to be inserted into the lumen 60 of the stent 30 and artery 58 without colliding with the proximal end 54 of the stent 40 or becoming entrapped between the outer surface of the stent 40 and the inner surface 64 of the artery 58.

Other variations of the described teachings will occur to those skilled in the art given the benefit of the described teachings. The following claims define the scope of the invention.

What is claimed is:

1. A stent comprising:
a substantially cylindrical expandable structure comprising a plurality of interconnected elements; and
A filter mesh operatively connected to at least some elements from said plurality of interconnected elements.

2. The stent of claim 1 wherein said filter mesh is operatively connected on to an outer substantially cylindrical surface of said substantially cylindrical expandable structure.

3. The stent of claim 2 wherein said filter mesh comprises a shape memory alloy.

4. The stent of claim 1 wherein said filter mesh is operatively connected onto an inner substantially cylindrical surface of said substantially cylindrical expandable structure.

5. The stent of claim 4 wherein said filter mesh comprises a shape memory alloy.

6. The stent of claim 1 wherein said filter mesh is interdigitated with said plurality of interconnected elements.

7. The stent of claim 6 wherein said filter mesh comprises a shape memory alloy.

8. The stent of claim 1 wherein said substantially cylindrical expandable structure comprises a shape memory alloy.

9. The stent of claim 1 wherein said substantially cylindrical expandable structure has two ends; a locus of points at one of said two ends defining a surface, said surface being beveled with respect to a central axis of said substantially cylindrical expandable structure.

10. The stent of claim 9 wherein a locus of points at another one of said two ends defines another surface, said another surface being beveled with respect to the central axis of said substantially cylindrical expandable structure.

11. The stent of claim 10 wherein an angle between a normal to said surface and said central axis is different from an angle between a normal to said another surface and said central axis.

12. The stent of claim 1 wherein said substantially cylindrical expandable structure is covered with a synthetic material.

13. A method for protecting from embolization, the method comprising the steps of:
providing a stent comprising a substantially cylindrical expandable structure having a plurality of interconnected elements and a filter mesh operatively connected to at least some of elements from the plurality of interconnected elements; and
delivering the stent to a predetermined intraluminal location.

14. The method of claim 13 wherein the step of delivering the stent to a predetermined intraluminal location comprises the steps of:
mounting the stent, onto an inflatable balloon on a distal end of a delivery catheter;
introducing the delivery catheter with the stent mounted on the inflatable balloon within a patience vasculature;
advancing the delivery catheter with the stent mounted on the inflatable balloon over a guide wire to a predetermined position in a lumen;
expanding the balloon, causing the stent to expand against an inner surface of the lumen; and
contracting the balloon and removing the catheter;
whereby the filter mesh substantially provides protection from embolization.

15. A method for protecting from embolization when placing an angled catheter into a lumen of a stented vessel, the method comprising the step of:
providing a stent comprising a substantially cylindrical expandable structure having a plurality of interconnected elements and a filter mesh operatively connected to at least some of elements from the plurality of interconnected elements, the stent having a proximal end, a locus of points at the proximal end defining a surface, the surface being beveled with respect to a central axis of the stent, the proximal end comprising a beveled end; and
inserting the angled catheter into a lumen of the stent.
